(19) United States
(12) Patent Application Publication
(10) Pub. No.: US 2025/0125699 A1
(43) Pub. Date: Apr. 17, 2025

(54) MAGNETIC DRIVE SYSTEM AND BREAST PUMP

(71) Applicant: Shenzhen TPH Technology Co.,Ltd., Shenzhen (CN)

(72) Inventor: Xianzi CHEN, Shenzhen (CN)

(21) Appl. No.: 18/984,997

(22) Filed: Dec. 17, 2024

Related U.S. Application Data
(63) Continuation of application No. PCT/CN2024/123699, filed on Oct. 9, 2024.

(30) Foreign Application Priority Data
Sep. 29, 2024 (CN) 202411374186.7

(51) Int. Cl. H02K 33/16; H02K 1/34; H02K 7/14

(52) U.S. Cl. CPC H02K 33/16; H02K 1/34; H02K 7/14

(57) ABSTRACT

A magnetic drive system includes a flexible component, a mounting structure, a first magnetic component and a second magnetic component. The flexible component is provided on the mounting structure, and the flexible component is in sealed connection with the mounting structure. The mounting structure is provided with an air hole. The first magnetic component is arranged in the flexible component. The second magnetic component is arranged opposite to the first magnetic component. N and S poles of the second magnetic component are configured to be interchanged. A breast pump including the magnetic drive system is also provided.

### CROSS-REFERENCE TO RELATED APPLICATIONS

This application is a continuation of International Patent Application No. PCT/CN2024/123699, filed on Oct. 9, 2024, which claims the benefit of priority from Chinese Patent Application No. 202411374186.7, filed on Sep. 29, 2024. The content of the aforementioned application, including any intervening amendments thereto, is incorporated herein by reference in its entirety.

### TECHNICAL FIELD

This application relates to breast pumps, and more particularly to a magnetic drive system and a breast pump.

### BACKGROUND

Breast pump is designed to express breast milk accumulated in the breast. The breast pump is generally suitable for the case that the baby is unable to be breastfed directly, or the mother has problems with her nipples, or other cases that it is not convenient for the mother to breastfeed her baby.

Most of the traditional breast pumps adopt a vacuum pump to directly produce the negative pressure or adopt a mechanical structure to drive the flexible membrane to deform to produce the negative pressure, so as to generate the suction for milk pumping. However, these methods are generally accompanied by the generation of strong noise, which will affect the user's mood and reduce the use comfortability. Therefore, it is urgent to provide a novel drive system to solve the problem of large noise during the operation of the breast pump.

### SUMMARY

An object of this application is to provide a magnetic drive system and a breast pump to solve the above problems in the prior art.

In a first aspect, this application provides a magnetic drive system comprises:
a flexible component;
a mounting structure;
a first magnetic component; and
a second magnetic component;
wherein the flexible component is provided on the mounting structure, and the flexible component is in sealed connection with the mounting structure; and the mounting structure is provided with an air hole group;
the first magnetic component is arranged in the flexible component; and
the second magnetic component is arranged opposite to the first magnetic component; and the second magnetic component is configured to alternately generate N pole and S pole.

In a second aspect, this application provides a breast pump, comprising the magnetic drive system above.

This application has the following beneficial effects.

This application changes a traditional milk pumping form of a vacuum pump and an electromagnetic valve to an electromagnetic milk pumping form. The second magnetic component can alternately generate N pole and S pole, so as to drive the flexible component to deform to generate a negative pressure for the milk pumping. The vacuum pump and the electromagnetic valve are cancelled to eliminate a noise source, which greatly reduces noise and even reaches “zero” noise, eliminating an influence on moods of mother and improving a comfort.

### BRIEF DESCRIPTION OF THE DRAWINGS

In order to illustrate the technical solutions of this application or the prior art more clearly, the accompanying drawings required in the description of embodiments or the prior art will be briefly introduced below. It is obvious that the following accompanying drawings only show some embodiments of this application, and for those of ordinary skill in the art, other relevant accompanying drawings can also be obtained according to these drawings without making creative effort.

FIG. 1 schematically shows a whole structure of a breast pump magnetically driven to generate a negative pressure according to an embodiment of the present disclosure.

FIG. 2 is an exploded view of the breast pump according to an embodiment of the present disclosure.

FIG. 3 schematically shows an assembly structure of the breast pump according to an embodiment of the present disclosure.

FIG. 4 schematically shows a sectional structure of the breast pump according to an embodiment of the present disclosure.

FIG. 5 is an enlarged view of portion “A” in FIG. 4.

FIG. 6 is a structural diagram of a connecting part of the breast pump according to an embodiment of the present disclosure.

FIG. 7 is a structural diagram of a cover of the breast pump according to an embodiment of the present disclosure.

FIG. 8 is a structural diagram of a flexible component of the breast pump according to an embodiment of the present disclosure.

FIG. 9 is an inner structural diagram of the flexible component according to an embodiment of the present disclosure.

FIG. 10 schematically shows a control system of the breast pump according to an embodiment of the present disclosure.

FIG. 11 is a flow diagram of a control method of the breast pump according to an embodiment of the present disclosure.

In the Figures: 1, first shell; 11, breast shield; 111, receptacle; 112, milk collection cavity; 12, cover; 121, mounting part; 122, convex ring; 123, mounting groove; 124, through hole; 125, air drain; 126, first quick-release structure; 127, liquid outlet; 2, second shell; 21, first half shell; 211, button; 212, charging port; 22, second half shell; 221, second quick-release structure; 3, connecting part; 31, mounting cylinder; 311, limiting component; 32, first connecting tube; 33; second connecting tube; 34, mounting structure; 341, first clamping portion; 4, flexible component; 4a, assembly part; 4b, deforming part; 41, first magnetic component; 42, second clamping portion; 43, annular groove; 44, bulge; 45, third magnetic component; 46, trough; 47, air hole group; 5, second magnetic component; 6, control panel; 7, power supply device; and 8, one-way valve.

The realization of objectives, functional characteristics and advantages of the present disclosure will be further described with reference to the accompanying drawings.

### DETAILED DESCRIPTION OF EMBODIMENTS

The technical solutions of the embodiments of the present disclosure will be clearly and completely described with reference to the accompanying drawings of the embodiments of the present disclosure. It is obvious that described herein are only some embodiments of the present disclosure, rather than all embodiments. Based on the embodiments of the present disclosure, other embodiments obtained by those of ordinary skill in the art without making creative effort shall fall within the scope of the present disclosure.

It should be noted that the terms, such as “up”, “down”, “left”, “right”, “front”, “rear” and other directional indications used herein, are only used for illustrating relative position relationship and motion between components in a specific state (as shown in the accompanying drawings). If the specific state changes, the directional indication accordingly changes.

In addition, the terms “first” and “second” are only used for distinguishment rather than indicating or implying the relative importance or implicitly specifying the number of technical features indicated. Therefore, a feature defined with “first” or “second” may explicitly or implicitly indicates the inclusion of at least one of such features. Besides, the term “and/or” used herein includes three solutions, for example, “A” and/or “B” includes solution “A”, solution “B”, and a combination thereof. Technical solutions of embodiments can be combined with each other as long as the combined solution can be implemented by those skilled in the art. When a combination of the technical solutions is contradictory or cannot be realized, it should be considered that such a combination does not exist, and is not within the scope of the present disclosure.

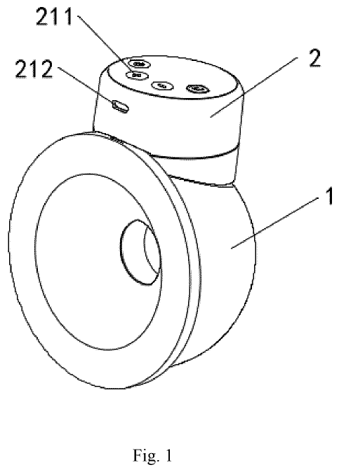

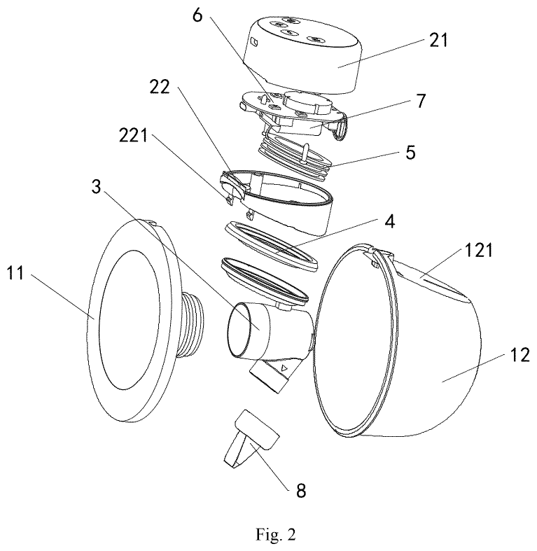

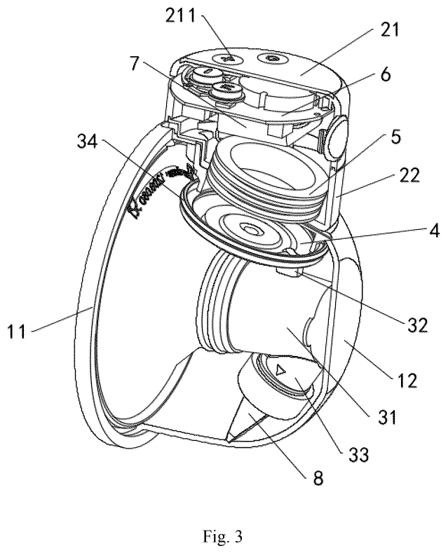

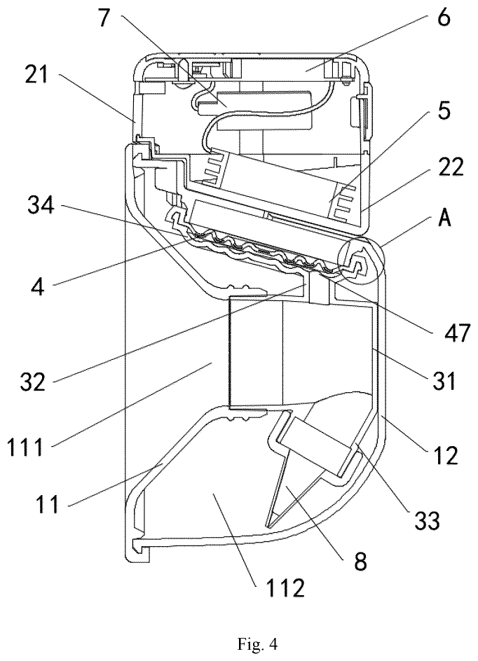

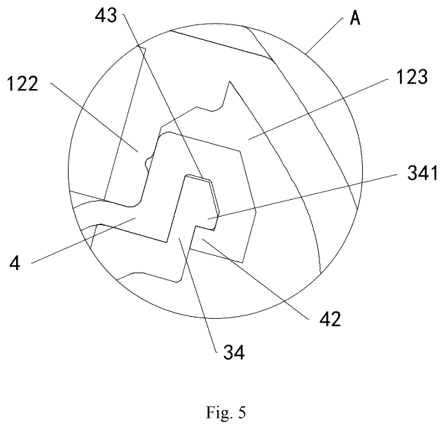

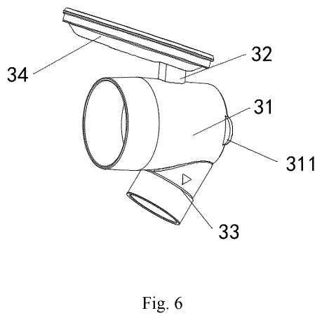

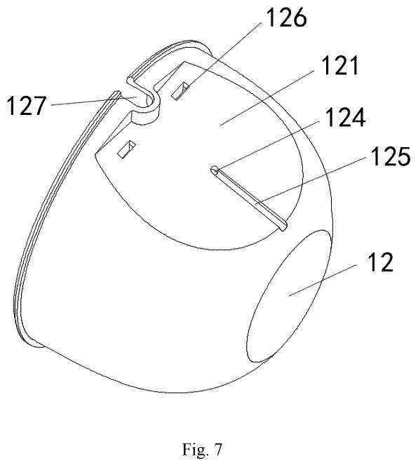

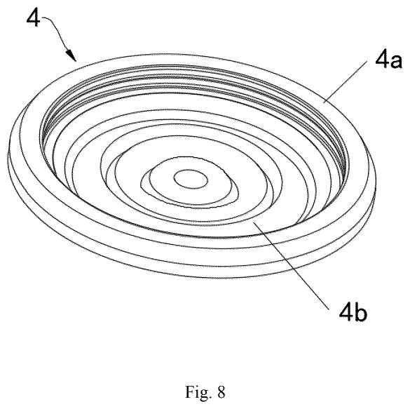

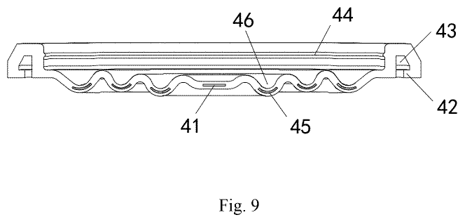

Referring to FIGS. 1-9, the embodiments of this application provide a breast pump magnetically driven to generate a negative pressure, including a breast shield 11, a flexible component 4 and a second magnetic component 5. The breast shield 11 is configured to form a receptacle 111. The receptacle 111 is configured to fit and at least partially accommodate a breast of the user without a gap to form a pressure-tight cavity.

The flexible component 4 is provided with a first magnetic component 41. At least part of the flexible component 4 is configured to be connected with the pressure-tight cavity and form a part of a wall of the pressure-tight cavity.

The first magnetic component 41 is arranged opposite to the second magnetic component 5. The second magnetic component 5, through a magnetic force is configured to drive the first magnetic component 41 to operate and drive the flexible component 4 to deform, so that the pressure-tight cavity is configured to generate a negative pressure.

In this embodiment, an inner wall of the breast shield 11 is configured to fit the breast of the user. After fit of the breast of the user and the inner wall of the breast shield 11, the pressure-tight cavity enclosed by the breast shield 11 and the flexible component 4 is in a pressure-tight state.

The first magnetic component 41 is arranged opposite to the second magnetic component 5, and a position of the first magnetic component 41 and a position of the second magnetic component 5 can be exchanged. It should be noted that magnetic components herein can be selected from components with the magnetic force and materials that can be guided to by the magnetic force.

In other embodiments, the first magnetic component 41 includes but is not limited to a permanent magnet, and can be in the shape of ring, block or strip. The second magnetic component 5 is an electromagnetic coil, and can be in the shape of ring, block or strip.

During operation, the inner wall of the breast shield 11 is configured to fit the breast of the user, and then the second magnetic component 5 is started to generate the magnetic force to drive the first magnetic component 41 to operate. In a case that a magnetic pole of the second magnetic component 5 and a magnetic pole of the first magnetic component 41 are different, the first magnetic component 41 and the second magnetic component 5 are configured to generate the magnetic force. Under the action of the magnetic force, the flexible component 4 is driven to deform. At this time, the negative pressure is generated at an interior of the pressure-tight cavity. With the cooperation of the breast shield 11, a pressure is applied on the breast of the user to pump the milk breast. In a case that the magnetic pole of the second magnetic component 5 and the magnetic pole of the first magnetic component 41 are the same, the first magnetic component 41 and the second magnetic component 5 are configured to generate a repulsive force to drive the flexible component 4 to return. At this time, the negative pressure at the interior of the pressure-tight cavity is eliminated.

This application changes a traditional milk pumping form of a vacuum pump and an electromagnetic valve to an electromagnetic milk pumping form, which greatly reduces the weight of the breast pump, and is easier to carry. In addition, the vacuum pump and the electromagnetic valve are cancelled to eliminate a noise source, which greatly reduces noise and even reaches “zero” noise, eliminating an influence on moods of mother and improving a comfort.

In addition, this application replaces components, such as the vacuum pump and the electromagnetic valve with a magnetic suction method, reducing mounting assemblies (components, such as the vacuum pump, the electromagnetic valve and sealing ring), and effectively reducing the cost.

In an embodiment, an interior of the flexible component 4 is provided with at least one third magnetic component 45. The at least one third magnetic component 45 is configured to be arranged around a periphery of the first magnetic component 41.

In the embodiment, the at least one third magnetic component 45 is pre-embedded at the interior of the flexible component 4 to further improve magnetic properties, so as to improve efficiency and reduce energy loss; where the at least one third magnetic component 45 is a neodymium magnet.

Each of the at least one third magnetic component 45 is ring-shaped. In a case that the number of the at least one third magnetic component 45 is two or more, the two or more third magnetic components 45 are arranged in an ascending manner in diameter from inside to outside along a radial direction of the flexible component 4.

In an embodiment, the pressure-tight cavity is communicated with a milk collection assembly, and the milk collection assembly is configured to collect and store the breast milk.

In this embodiment, a container, as long as it can collect and store the breast milk, is configured as the milk collection assembly, such as a feeding bottle and a cup-shaped container.

In an embodiment, the milk collection assembly includes a cover 12. The cover 12 and the breast shield 11 are configured to form a first shell 1. An interior of the first shell 1 is provided with a milk collection cavity 112. A diameter of a first end of the breast shield 11 is smaller than that of a second end of the breast shield 11, and the first end of the breast shield 11 is configured to extend into the milk collection cavity 112.

In this embodiment, the breast shield 11 is in sealed connection with the cover 12. The cover 12 is similar to a hemispherical shape. An open edge of the cover 12 is provided with a liquid outlet 127, and the liquid outlet 127 is configured to pour the breast milk after milk pumping.

In an embodiment, the breast pump further includes a second shell 2. The second magnetic component 5 is fixed on the second shell 2. The second magnetic component 5 is configured to be switched between N pole and S pole.

In this embodiment, the second magnetic component 5 is configured to be switched between the N pole and S pole to drive the flexible component 4 to realize a reciprocating movement of deformation and return and form a stable function of suction and loosening, so that a continuous and stable milk pumping process is achieved.

In an embodiment, the first magnetic component 41 is configured to be S pole. In a case that the second magnetic component 5 is configured as the N pole, the first magnetic component 41 and the second magnetic component 5 generate the magnetic force to drive the flexible component 4 to deform and generate the negative pressure inside the pressure-tight cavity to, so as to realize the milk expressing. In a case that the second magnetic component 5 is configured as the S pole, the first magnetic component 41 and the second magnetic component 5 generate the repulsive force to push the flexible component 4 to return, and the negative pressure at the interior of the pressure-tight cavity is eliminated.

Negative pressure conduction made by the vacuum pump has delay, its efficiency can only be improved depend on a resilience of an original material, and it is necessary to evacuate air in the first shell 1, the second shell 2 and the flexible component 4 to generate the negative pressure to a human body. The magnetic suction method is adopted, which can instantly change a direction and magnitude of a magnetic field. In addition, the repulsive force can drive the flexible component 4 to quickly return, which can reduce the delay and greatly improve the efficiency of the milk pumping.

In an embodiment, the breast pump further includes a connecting part 3. The breast shield 11 is connected with the flexible component 4 through the connecting part 3 to form the pressure-tight cavity. An interior of the connecting part 3 has a hollow structure. An end of the connecting part 3 is provided with an opening of the connecting part 3, and the first end of the breast shield 11 is connected with the end of the connecting part 3 provided with the opening of the connecting part 3.

In this embodiment, the connecting part 3 includes but is not limited to a three-way valve. The breast shield 11 is configured to be in detachable and sealed connection with the connecting part 3. The flexible component 4 is configured to be in detachable and sealed connection with the connecting part 3.

In an embodiment, the connecting part 3 is communicated with the milk collection assembly. A one-way valve 8 is provided between the connecting part 3 and the milk collection assembly.

In this embodiment, the one-way valve 8 is provided. When the negative pressure is generated in the pressure-tight cavity, the one-way valve 8 is configured to prevent the breast milk in the milk collection assembly from returning to the pressure-tight cavity; where the one-way valve 8 is a duckbill valve. Specifically, When the negative pressure is generated in the pressure-tight cavity, an internal pressure of the pressure-tight cavity is less than an external pressure, so that the one-way valve 8 is deformed to close. After that, the one-way valve 8 is configured to prevent air leakage, increase air tightness, and improve the milk pumping effect. When the negative pressure is eliminated, the one-way valve 8 returns and opens, a pumped breast milk flows form the one-way valve 8 to the cover 12.

In an embodiment, the connecting part 3 is provided with a mounting structure 34. The mounting structure 34 is communicated with the connecting part 3. The flexible component 4 is arranged in the mounting structure 34.

In this embodiment, the mounting structure 34 is a suction bowl. The mounting structure 34 further includes structures of different shapes, such as round, square, or flat structures, which are included in the scope of the mounting structure 34 of the present disclosure as long as a fixation of the flexible component 4 can be realized. An edge of the flexible component 4 is in sealing connection or interference fit with an edge of the mounting structure 34, so that an interior of the mounting structure 34 is in a relative seal state. In an embodiment, the shape of the flexible component 4 matches that of the mounting structure 34.

A bottom of the mounting structure 34 is provided with an air hole group 47. The air hole group 47 is configured to connect the interior of the mounting structure 34 and the connecting part 3.

In an embodiment, the mounting structure 34 is configured to fit to the flexible component 4.

In this embodiment, the mounting structure 34 is configured to fit to the flexible component 4, so that there is no gap between the mounting structure 34 and the flexible component 4. During the milk pumping, if the breast milk is accidentally sucked into the mounting structure 34, the flexible component 4 is configured to return and discharge the breast milk in the mounting structure 34 in time, so that the breast milk will not remain in the mounting structure 34.

In an embodiment, an inner wall of the cover 12 is provided with a convex ring 122. The cover 12 is connected with the mounting structure 34 through the convex ring 122. The convex ring 122 extends from an opening of the mounting structure 34 to the mounting structure 34. The convex ring 122 and the mounting structure 34 are configured to clamp the flexible component 4.

In this embodiment, the shape of the convex ring 122 is consistent with a cup shape of the mounting structure 34. A side of the convex ring 122 and the inner wall of the cover 12 are configured to form a mounting groove 123. After mounting of the flexible component 4 and the mounting structure 34, the mounting structure 34 mounted with the flexible component 4 is configured to be installed into the mounting groove 123. At this time, the convex ring 122 is configured to be installed into the mounting structure 34 to finish the mounting. The convex ring 122 also has a positioning function.

In addition, in a case that a distance between an outer side wall of the convex ring 122 and an inner wall of the mounting structure 34 is larger than a thickness of the flexible component 4, if the flexible component 4 deforms, the convex ring 122 is configured to improve a connection strength of a connection of the flexible component 4 and the mounting structure 34, so as to prevent a sealing of the connection of the flexible component 4 and the mounting structure 34 from being reduced due to the deformation of the flexible component 4. In a case that the distance between the outer side wall of the convex ring 122 and the inner wall of the mounting structure 34 is slightly less than the thickness of the flexible component 4, the convex ring 122 is configured to improve the connection strength of the connection of the flexible component 4 and the mounting structure 34. The convex ring 122 and the mounting structure 34 are configured to clamp the flexible component 4 to form an interference fit, so that the flexible component 4 is configured to closely fit the inner wall of the mounting structure 34, which can further improve the sealing.

In an embodiment, the cover 12 is provided with a mounting part 121. The mounting part 121 is configured as a clamping groove or a magnetic structure. The cover 12 is in detachable connection with the second shell 2 through the mounting part 121.

In an embodiment, a surface of the cover 12 is provided with a through hole 124. The through hole 124 is communicated with a cavity formed by the cover 12 and the flexible component 4.

In this embodiment, the through hole 124 is provided, so that an air pressure of the cavity formed by the cover 12 and the flexible component 4 is consistent with the atmospheric pressure, and the flexible component 4 returns.

In an embodiment, the surface of the cover 12 is provided with an air drain 125. The air drain 125 is communicated with the through hole 124.

In this embodiment, when the through hole 124 is arranged on the mounting part 121, an interior of the cover 12 can also be communicated to the outside through the air drain 125, so that the air pressure of the cavity formed by the cover 12 and the flexible component 4 is consistent with the atmospheric pressure, which is not affected by the second shell 2.

In an embodiment, the second magnetic component 5 is configured to be controlled by a control mechanism. The control mechanism is arranged in the second shell 2.

In this embodiment, the control mechanism is configured to control the magnetic pole of the second magnetic component 5, so as to realize a constant alternation of the N pole and S pole of the magnetic pole of the second magnetic component 5.

In an embodiment, the control mechanism includes a control panel 6 and a power supply device 7. The control panel 6 is electrically connected to the second magnetic component 5. The power supply device 7 is electrically connected to the control panel 6.

In this embodiment, the power supply device 7 is a storage battery, and the number of the storage battery is one. In other embodiments, the number of the storage battery is two or more. The power supply device 7 is configured to supply power to the second magnetic component 5 and the control panel 6.

In an embodiment, when the power supply device 7 supplies power to the second magnetic component 5, the second magnetic component 5 is configured to generate the magnetic field through a current to generate the magnetic pole of the second magnetic component 5. A direction and magnitude of the current is controlled by the control panel 6, so as to control a magnetic pole conversion and a strength of the magnetic field of the second magnetic component 5.

In an embodiment, the first magnetic component 41 is configured as the S pole. In a case that the direction of the current makes the second magnetic component 5 is configured as the N pole, the first magnetic component 41 and the second magnetic component 5 generate the magnetic force, so that the negative pressure is generated at the interior of the pressure-tight cavity, to realize milk pumping. In a case that the direction of the current makes the second magnetic component 5 is configured as the S pole, the first magnetic component 41 and the second magnetic component 5 generate the repulsive force to push the flexible component 4 to return in the direction away from the second shell 2, and the negative pressure at the interior of the pressure-tight cavity is eliminated.

In an embodiment, the current can be accurately controlled. Through controlling the magnitude of the current, the strength and magnitude of the magnetic field and a suction magnitude are accurately controlled, so as to realize the adjustment of the negative pressure, which effectively improves the comfort.

In an embodiment, the second shell 2 includes a first half shell 21 and a second half shell 22. The first half shell 21 is in detachable connection with the second half shell 22. A lower end of the second half shell 22 is provided with a second quick-release structure 221. The lower end of the second half shell 22 is detachably mounted on the mounting part 121 through the second quick-release structure 221.

In this embodiment, the first half shell 21 is in detachable connection with the second half shell 22, and the lower end of the second half shell 22 is detachably mounted on the mounting part 121, so that the assembly and disassembly of the breast pump are convenient and fast.

In this embodiment, the mounting part 121 is provided with a first quick-release structure 126 corresponding to the second quick-release structure 221. In a case that the first quick-release structure 126 is a snap-fit hole, the second quick-release structure 221 is a snap fastener. In addition, the first quick-release structure 126 and the second quick-release structure 221 can be magnetic elements.

In an embodiment, the first half shell 21 is provided with a plurality of buttons 211.

In this embodiment, the plurality of buttons 211 are provided to send different control instructions to a controller to achieve different functions.

In this embodiment, the plurality of buttons 211 include a power button, a level selection button, a pause button and a mode switch button; where the power button is configured to control start and stop of the breast pump; the level selection button includes a “+” button configured to raise a suction level of the breast pump and a “−” button configured to lower the suction level of the breast pump; and the pause button is configured to control pause of operation of the breast pump. The breast pump of the present disclosure includes a massage mode, a pumping mode, a stimulus mode and an automatic mode. The mode switch button is configured to switch the four modes of the breast pump.

In an embodiment, the cover 12 is transparent. An outer surface of the cover 12 is provided with a plurality of graduations.

In this embodiment, the cover 12 is transparent to observe breast milk collection and internal cleaning at every moment, which facilitates timely cleaning and prevents bacterial growth.

In addition, the outer surface of the cover 12 is provided with the plurality of graduations, and is also provided with different labels, so as to measure the amount of breast milk.

In an embodiment, the first half shell 21 is provided with a display screen. The display screen is electrically connected with the control panel 6.

in this embodiment, the display screen is configured to display the negative pressure of the breast pump, a power level, a charging state, time and a suction level information.

In an embodiment, the connecting part 3 includes a mounting cylinder 31, a first connecting tube 32 and a second connecting tube 33. An end of the mounting cylinder 31 is provided with an opening of the mounting cylinder 31. The end of the mounting cylinder 31 with the opening of the mounting cylinder 31 is in detachable connection with the first end of the breast shield 11. A first end of the mounting cylinder 31 is communicated with the air hole group 47 of the mounting structure 34 through the first connecting tube 32. A second end of the mounting cylinder 31 is communicated with the cover 12 through the second connecting tube 33. The one-way valve 8 is sleevedly provided on an end of the second connecting tube 33 away from the mounting cylinder 31, and the first connecting tube 32 is located above the second connecting tube 33.

In this embodiment, the end of the mounting cylinder 31 with the opening of the mounting cylinder 31 is in sealing connecting or interference connection with the first end of the breast shield 11 to realize relative sealing. In this embodiment, a cross-section shape of the mounting cylinder 31 is round. In other embodiments, the cross-section shape of the mounting cylinder 31 can be polygonal or in an irregular shape. The second connecting tube 33 is provided, so that the breast milk in the mounting cylinder 31 can flow through the second connecting tube 33 to the cover 12 for storage.

In an embodiment, the connecting part 3 is provided with a limiting component 311. The limiting component 311 is configured to limit a relative movement between the connecting part 3 and the milk collection assembly.

In this embodiment, the limiting component 311 is a semi-circular sheet, and the number of the limiting component 311 is at least two. At least two limiting components 311 are arranged on two sides of the connecting part 3, respectively. The limiting component 311 is configured to limit and position the connecting part 3 when the connecting part 3 and the cover 12 are assembled.

In an embodiment, an outer edge of the opening of the mounting structure 34 is provided with a first clamping portion 341. A cup wall of the mounting structure 34 and the first clamping portion 341 are configured to form a hook structure, and an upper end of the hook structure is configured to be inclined towards a bottom of the cover 12. An outer edge of the flexible component 4 is configured to bend downward to form an annular groove 43. The outer edge of the flexible component 4 is provided with a second clamping portion 42 extending to an interior of the annular groove 43. The first clamping portion 341 is configured to be in snap-fit connection with the second clamping portion 42 in response to a case that the first clamping portion 341 is inserted into the annular groove 43.

In this embodiment, the second clamping portion 42 is arranged on an opening of the annular groove 43 to form a narrowing shape. During mounting, the first clamping portion 341 is configured to be inserted into the annular groove 43, and then the second clamping portion 42 is in snap-fit connection with the first clamping portion 341, which is not easily separated. Under the action of an inherent elasticity of the flexible component 4 and the hook structure, the flexible component 4 is tightened, so that the mounting structure 34 is closely connected with the flexible component 4 to realize a better sealing effect.

The present disclosure also provides a flexible membrane for a negative pressure generated by magnetic drive. The flexible membrane is the flexible component 4 of the breast pump, and includes an assembly part 4a and a deforming part 4b. The assembly part 4a is configured to position and fix the flexible membrane. The deforming part 4b and the assembly part 4a are integrally formed. The deforming part 4b is in sealed connection with the assembly part 4a. The assembly part 4a is provided on an edge of the deforming part 4b.

In this embodiment, the deforming part 4b is a round sheet. In an embodiment, the deforming part 4b is in the shape of polygon or other irregular shapes. The assembly part 4a is in the shape of ring, and its shape matches that of the deforming part 4b. The deforming part 4b and assembly part 4a are integrally formed through injection molding. The deforming part 4b and the assembly part 4a are configured to form a cup structure. The first magnetic component 41 is in the shape of block, and is arranged at a center of the deforming part 4b. The assembly part 4a and deforming part 4b are made of latexes. An edge of the assembly part 4a is provided with a groove structure, and the groove structure is the annular groove 43. The flexible membrane is mounted on the mounting structure 34 through the groove structure, so as to facilitate quick connection of the flexible membrane and the suction bowl, which is convenient for assembly. In an embodiment, the groove structure is located at an outer side of the deforming part 4b, and an opening of the groove structure is downward.

The second clamping portion 42 is arranged on an inner wall of the opening of the annular groove 43, and protrudes towards the deforming part 4b. In addition, the second clamping portion 42 is located on a side wall of the annular groove 43 away from the deforming part 4b.

By inlaying first magnetic component 41 in the deforming part 4b, the deforming part 4b can realize the deformation effect through the magnetic drive without using the vacuum pump or mechanical structure drive, which avoids the generation of noise from the source and does not affect the user's mood.

In an embodiment, the at least one third magnetic component 45 is inlaid in the deforming part 4b, and is located on the periphery of the first magnetic component 41.

In this embodiment, in a case that the number of the at least of third magnetic component 45 is in plurality, a plurality of third magnetic components 45 are arranged in an ascending manner in diameter from inside to outside along a radial direction of the deforming part 4b.

In an embodiment, the deforming part 4b has a wave-shaped cross-section.

In this embodiment, the deforming part 4b in wave-shaped is configured to improve a resilience of the deforming part 4b; where the at least of third magnetic component 45 is arranged at a trough 46 of the deforming part 4b. In an embodiment, a cross-section shape of the at least one third magnetic component 45 is arc-shaped, so that the at least one third magnetic component 45 matches the trough 46 of the deforming part 4b.

A molding process of the flexible membrane (that is, the flexible component 4) is as follows.

First, an integrated assembly part 4a of the flexible membrane is obtained through the injection molding.

Then, the first magnetic component 41 and the at least one third magnetic component 45 are inlaid in the deforming part 4b. Specifically, through comolding, the first magnetic component 41 and the at least one third magnetic component 45 are formed at a suction bowl membrane. At this time, a neodymium alloy at the assembly part 4a of the flexible membrane does not produce magnetism. After the comolding, the assembly part 4a of the flexible membrane (inlaid with the first magnetic component 41 and the at least one third magnetic component 45) is placed in a fixed position in a jig, so that it does not move freely. A suction bowl membrane assembly placed in the jig (inlaid with the first magnetic component 41 and the at least one third magnetic component 45) is placed in a magnetizing machine to magnetize. Then the assembly part 4a of the flexible membrane of the present disclosure is obtained.

Specific steps for preparing the assembly part 4a of the flexible membrane are as follows.
(S1) Greasy dirt and dust on a surface of the neodymium alloy (not magnetized) are cleaned.
(S2) A treating agent for bonding metal with silicone is applied on the surface of the neodymium alloy followed by dry processing according to instructions of the treating agent.
(S3) The neodymium alloy is placed in a mold for comolding to obtain a silica gel base.
(S4) The silica gel base is cleaned to remove surface greasy dirt and dust.
(S5) The treating agent is applied on the surface of the silica gel base followed by dry processing according to instructions of the treating agent.
(S6) The silica gel base after dry processing is placed in the mold for comolding with the flexible membrane to a semi-finished flexible membrane (inlaid with the neodymium alloy).
(S7) The semi-finished flexible membrane (inlaid with the neodymium alloy) is placed in the jig, so that the semi-finished flexible membrane does not move freely during magnetization, followed by placing in the magnetizing machine to obtain a magnetized flexible membrane.

In an embodiment, the flexible component 4 is provided with a bulge 44. The bulge 44 is arranged on side wall of the assembly part 4a near the deforming part 4b and away from the annular groove 43.

In this embodiment, after assembly of the convex ring 122 and the suction bowl, the bulge 44 is configured to be in interference fit with the side wall of the convex ring 122 to improve sealing of a connection of the bulge 44 and the convex ring 122.

In an embodiment, the first magnetic component 41 and the at least one third magnetic component 45 are configured as the neodymium magnet.

In this embodiment, the neodymium magnet is prepared by magnetizing the neodymium alloy.

This application provides a magnetic drive system including components of the breast pump magnetically driven to generate a negative pressure in the embodiment above. The magnetic drive system includes the flexible component 4, the mounting structure 34, the first magnetic component 41 and the second magnetic component 5. The flexible component 4 is arranged on the mounting structure 34, and the flexible component 4 is in sealed connection with the mounting structure 34. The mounting structure 34 is provided with the air hole group 47. The first magnetic component 41 is arranged in the flexible component 4. The second magnetic component 5 is arranged opposite to the first magnetic component 41. The second magnetic component 5 is configured to be switched between N pole and S pole.

This application changes the traditional milk pumping form of the vacuum pump and the electromagnetic valve to the electromagnetic milk pumping form. The second magnetic component 5 is configured to alternately generate N pole and S pole, so as to drive the flexible component to deform to generate the negative pressure for the milk pumping. The vacuum pump and the electromagnetic valve are cancelled to eliminate the noise source, which greatly reduces noise and even reaches “zero” noise, eliminating an influence on moods of mother and improving the comfort.

In an embodiment, the magnetic force of the first magnetic component 41 and the second magnetic component 5 (that is, the electromagnetic coil) is calculated through design needs, and is expressed as:

P=F/S; and

F=P*S;

where P represents an intensity of pressure; F represents a pressure; and S represents a contact area.

According to a conversion of the air pressure and force, it can be known that 1 pa=1 N/m². In addition, 1 millimeter of mercury (mmHg)=133.3223684 pascal (Pa).

It can be known that an area of the mounting structure 34 is calculated and is plugged the formula above, and then a force required to pull the flexible component 4 of the mounting structure 34 upward is obtained.

Based on a Maxwell's equation for electromagnetic force, formulas are obtained as follows.

The electromagnetic force is expressed as:

$$F = {B}^{2} * S / 2 * {μ}^{0} = {φ}^{2} / 2 * μ0 * S ; \qquad ( 1 )$$

where B represents a magnetic flux density (unit: T); S represents a cross-sectional area of a magnetic circuit (unit: m²); φ represents magnetic flux through the cross-sectional area (unit: Wb); and μ⁰ represents a vacuum permeability (4π*10⁻⁷ Wb/A·m).

Without considering a magnetic leakage loss and an air gap loss of the connection, only a route of deformation action of the flexible component 4 is regarded as a main air gap. The magnetic flux density B at the first magnetic component 41 inlaid in the flexible component 4 is expressed as:

$$B = N * U * {μ}^{0} / R * g = N * I * {μ}^{0} / g ; \qquad ( 2 )$$

where N represents the number of the electromagnetic coil; I represents a current intensity (unit: A); U represents a supply voltage (unit: V); R represents a resistance of a winding coil (unit: Ω); g represents a width of the air gap (unit: m) (that is, a distance between the first magnetic component 41 in the flexible component 4 and a cross section of the electromagnetic coil).

Formula (2) is plugged in formula (1), and the electromagnetic force is expressed as:

$$F = {( N * I )}^{2} * {μ}^{0} * S / 2 * {g}^{2} . \qquad ( 3 )$$

Through the above designs, an area of the first magnetic component 41 can be determined, and is the same as the cross-sectional area of a magnetic circuit S. in addition, a distance between the first magnetic component 41 and a cross section of the second magnetic component 5 is calculated, and is represented as g. The number of the electromagnetic coil N and the current intensity I can be regarded as debug values. N*I is in direct proportion to F, so that in a case that F is larger than an upper pull force, a need of the negative pressure is met.

The present disclosure also provides a control method for the breast pump in the embodiment above. The control method includes the following stops.
(S1) The breast shield 11 is provided to form a first cavity, so that the first cavity is configured to fit and at least partially accommodate the breast of the user without the gap to form the pressure-tight cavity.
(S2) The flexible component 4 is provided, and is provided with the first magnetic component 41. At least part of the flexible component 4 is configured to be connected with the pressure-tight cavity and form the part of the wall of the pressure-tight cavity.
(S3) The second magnetic component 5 is provided. The second magnetic component 5 and the first magnetic component 41 are arranged relatively. The second magnetic component 5 is configured to control the first magnetic component 41 to drive the flexible component 4 to deform, so that the negative pressure is generated in the pressure-tight cavity to force the breast to lactate.

In this embodiment, this application changes the traditional milk pumping form of the vacuum pump and the electromagnetic valve to the electromagnetic milk pumping form, which greatly reduces the weight of the breast pump, and is easier to carry. In addition, the vacuum pump and the electromagnetic valve are cancelled to eliminate the noise source, and the second magnetic component 5 is used to control the first magnetic component 41 to drive the flexible component 4 to deform, so that the negative pressure is generated in the pressure-tight cavity to force the breast to lactate, which greatly reduces noise and even reaches “zero” noise, eliminating an influence on moods of mother and improving a comfort.

In an embodiment, the second magnetic component 5 is configured to be switched between N pole and S pole, so as to drive the first magnetic component 41 to drive the flexible component 4 to reciprocate.

In an embodiment, step (S1) includes the following steps.

The breast shield 11 is connected with the connecting part 3. The interior of the connecting part 3 has the hollow structure. The end of the connecting part 3 is provided with the opening of the connecting part 3, and the first end of the breast shield 11 is connected with the end of the connecting part 3 provided with the opening of the connecting part 3.

The connecting part 3 is provided with the suction bowl, and an interior of the suction bowl is communicated with the connecting part 3.

The flexible component 4 is provided in the suction bowl. The breast shield 11 and the flexible component 4 are configured to form the pressure-tight cavity through the connecting part 3.

In an embodiment, step (S3) further includes the following steps.

The second magnetic component 5 is fixed in the second shell 2, so that the second magnetic component 5 is connected with the control mechanism. The control mechanism is provided in the second shell 2.

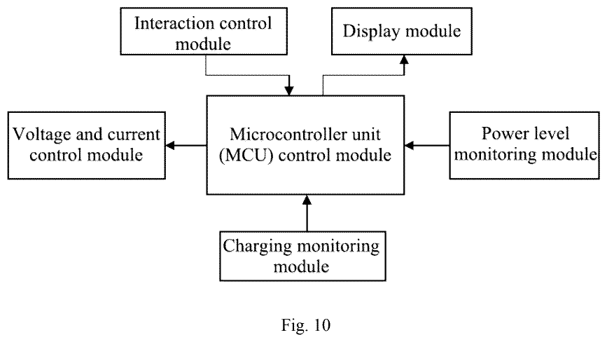

Referring to FIG. 10, the present disclosure provides a control system for breast pumping circuit of the breast pump magnetically driven to generate a negative pressure. The control system is configured to perform circuit control on the control method above. The control system includes a micro controller unit (MCU) control module, an interaction control module and a voltage and current control module. The MCU control module is configured to receive and send a signal. The interaction control module is configured to receive an operation signal of the plurality of buttons 211 from the user, and send the operation signal to the MCU control module. The voltage and current control module is configured to receive a control signal from the MCU control module to control the voltage and current of the electromagnetic coil to reach different suction levels. The voltage and current control module is also configured to send a suction level signal to the MCU control method.

In this embodiment, after user's operation on the plurality of buttons 211, the interaction control module is configured to send a corresponding operation signal of the plurality of buttons 211 to the MCU control module. The MCU control module is configured to receive the signal from the interaction control module and send the control signal to the voltage and current control module. The voltage and current control module, after receiving the control signal of the MCU control module, is configured to control the voltage and current of the electromagnetic coil according to instructions of the signal, so as to control the magnitude of the electromagnetic force to control a deformation degree of the flexible component 4 and then generate different suction forces.

In an embodiment, the control system further includes a power level monitoring module. The power level monitoring module is configured to monitor the power level of the power supply device 7 in real time, and send a data information to the MCU control module.

In this embodiment, the power level monitoring module is configured to monitor the power level of the storage battery in the breast pump, and send a monitoring data to the MCU control module.

In an embodiment, the control system further includes a charging monitoring module. The charging monitoring module is configured to detect an external charger and feed back to the MCU control module.

In this embodiment, when the storage battery of the breast pump is out of power, if the storage battery is charged, the charging monitoring module is configured to detect the charger and monitor the charging state.

In an embodiment, the control system further includes a display module. The display module is configured to receive the signal sent by the MCU control module from the voltage and current control module, the power level monitoring module and the charging monitoring module.

In this embodiment, the signal is sent to the MCU control module by the voltage and current control module, the power level monitoring module and the charging monitoring module followed by being sent to the display module by the MCU control module. The display module is configured to send the data information to the display screen to display.

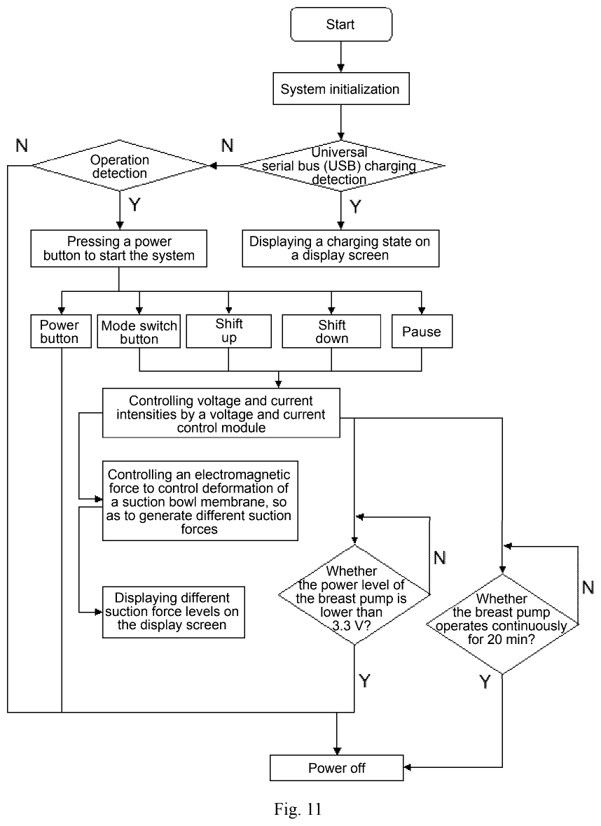

Referring to FIG. 11, the present disclosure provides a control method for breast pumping circuit of the breast pump magnetically driven to generate a negative pressure. The control method for breast pumping circuit is configured to control the control system for breast pumping circuit above. The control method for breast pumping circuit includes the following steps.
(S1) The breast pump is started, and the control system for breast pumping circuit is initialized.
(S2) The charging monitoring module detects whether the external charger is accessed. If yes, a signal of the charging state is sent to the display module to display.
(S3) The interaction control module determines whether there is an operation instruction of the plurality of buttons 211. If yes, the operation instruction is sent to the MCU control module; if no, the breast pump is automatically powered off.
(S4) The MCU control module receives the operation instruction from the interaction control module, and determines the operation instruction, and then sends to the voltage and current control module.
(S5) After receiving the operation instruction, the voltage and current control module controls the voltage and current of the electromagnetic coil, so as to control the magnitude of the electromagnetic force to generate different magnitudes of the suction force.

In this embodiment, before the operation, the control system for breast pumping circuit of the breast pump is needed to be initialized. After initialization, the user performs operation on the plurality of buttons 211. After the interaction control module determines there is the operation instruction of the plurality of buttons 211, the power button is pressed to start the magnetic drive system. Such design prevents the breast pump from starting by mistake. After the magnetic drive system is started, the user can perform control through the plurality of buttons 211. The instruction corresponding to the plurality of buttons 211 is sent, by the interaction control module to the MCU control module, and then is sent, by the MCU control module to the voltage and current control module. The voltage and current control module is configured to adjust the voltage and current of the electromagnetic coil to control the magnitude of the electromagnetic force to generate different magnitudes of the suction force, and feed back different suction levels to the display module to display.

In an embodiment, after the magnetic drive system is started, if the power button is pressed again, the breast pump will power off.

In an embodiment, operation instructions of the plurality of buttons 211 include a mode switch instruction, a shift up instruction, a shift down instruction and a pause instruction.

In this embodiment, the mode switch instruction, the shift up instruction, the shift down instruction and the pause instruction correspond to the mode switch button, the “+” button for raising the suction level, the “−” button for lowering the suction level and the pause button on the breast pump, respectively. Corresponding buttons are configured to be pressed to send corresponding instructions.

In an embodiment, after the operation of the breast pump, the control method for breast pumping circuit further includes the following steps. The power level monitoring module determines whether the voltage of the breast pump is lower than a minimum operation voltage, if yes, the breast pump automatically powers off, if not, the breast pump continues to operate.

In this embodiment, the minimum operation voltage is 3.3 V. If the voltage of the breast pump is lower than 3.3 V, the breast pump automatically powers off. If the voltage of the breast pump is higher than 3.3 V, the breast pump continues to operate.

In an embodiment, after the operation of the breast pump, the control method for breast pumping circuit further includes the following steps. The MCU control module determines whether a set continuous operation time is reached, if yes, the breast pump automatically powers off; if not, the breast pump continues to operate.

In this embodiment, the set continuous operation time is 20 min. If the set continuous operation time is reached, the breast pump automatically powers off. If the set continuous operation time is not reached, the breast pump continues to operate.

Described above are only preferred embodiments of the present disclosure, which are not intended to limit the disclosure. Under the sprits of this application, any equivalent replacements or direct/indirect application in other arts by utilizing the specification and accompanying drawings of this application shall fall within the scope of this application defined by the appended claims.

What is claimed is:

1. A magnetic drive system, comprising:
a flexible component;
a mounting structure;
a first magnetic component; and
a second magnetic component;
wherein the flexible component is provided on the mounting structure, and the flexible component is in sealed connection with the mounting structure; and the mounting structure is provided with an air hole group;
the first magnetic component is arranged in the flexible component; and
the second magnetic component is arranged opposite to the first magnetic component; and the second magnetic component is configured to be switched between N pole and S pole.

2. The magnetic drive system of claim 1, wherein the second magnetic component is an electromagnetic coil; the second magnetic component is configured to be controlled by a control mechanism; and the second magnetic component is electrically connected with the control mechanism.

3. The magnetic drive system of claim 2, wherein the control mechanism comprises a control panel and a power supply device; the control panel is electrically connected to the second magnetic component; and the power supply device is electrically connected to the control panel.

4. The magnetic drive system of claim 1, wherein an interior of the flexible component is provided with at least one third magnetic component, and the at least one third magnetic component is arranged around a periphery of the first magnetic component.

5. The magnetic drive system of claim 4, wherein a bottom of the flexible component has a wave-shaped cross-section; and the at least one third magnetic component is arranged at a trough of the flexible component.

6. The magnetic drive system of claim 1, wherein the mounting structure has a suction bowl-shaped structure;
an outer edge of an opening of the mounting structure is provided with a first clamping portion; a cup wall of the mounting structure and the first clamping portion are configured to form a hook structure; an outer edge of the flexible component is configured to bend downwards to form an annular groove; the outer edge of the flexible component is provided with a second clamping portion extending to an interior of the annular groove; and the first clamping portion is configured to be in snap-fit connection with the second clamping portion in response to a case that the first clamping portion is inserted into the annular groove.

7. The magnetic drive system of claim 4, wherein the at least one third magnetic component is provided in plurality; a plurality of third magnetic components are arranged spaced apart on the flexible component; and
each of the plurality of third magnetic components is ring-shaped; and the plurality of third magnetic components are arranged in an ascending manner in diameter from inside to outside along a radial direction of the flexible component.

8. The magnetic drive system of claim 6, wherein a bottom of the mounting structure is configured to fit a bottom of the flexible component.

9. A breast pump, comprising:
the magnetic drive system of claim 1.
